SEQUENCE LISTING (6 sequences)

SEQ ID NO: 1 - protein, 13 aa, Artificial Sequence
  Partial Sequence of Protein
  misc_feature (1)..(3): Xaa can be any naturally occurring amino acid
  misc_feature (5)..(6): Xaa can be any naturally occurring amino acid
  misc_feature (8)..(9): Xaa can be any naturally occurring amino acid
  misc_feature (11)..(12): Xaa can be any naturally occurring amino acid
XXXGXXGXXGXXG

SEQ ID NO: 2 - protein, 10 aa, Artificial Sequence
  Partial Sequence of Protein
  misc_feature (2)..(3): Xaa can be any naturally occurring amino acid
  misc_feature (5)..(6): Xaa can be any naturally occurring amino acid
  misc_feature (8)..(9): Xaa can be any naturally occurring amino acid
GXXGXXGXXG

SEQ ID NO: 3 - protein, 12 aa, Artificial Sequence
  Partial Sequence of Protein
VPGPMGPSGPRG

SEQ ID NO: 4 - protein, 8 aa, Artificial Sequence
  Partial Sequence of Protein
MGPSGPRG

SEQ ID NO: 5 - protein, 14 aa, Artificial Sequence
  Partial Sequence of Protein
GPGPMGLMGPRGPP

SEQ ID NO: 6 - protein, 8 aa, Artificial Sequence
  Partial Sequence of Protein
LMGPRGPP

(19) United States
(12) Patent Application Publication
(10) Pub. No.: US 2021/0047385 A1
(43) Pub. Date: Feb. 18, 2021

(54) NERVE CELL CULTURE MATERIAL AND THERAPEUTIC AGENT FOR NERVE DAMAGE

(71) Applicants: KINKI UNIVERSITY, OSAKA (JP); AINO UNIVERSITY, Osaka (JP); FOUNDATION FOR BIOMEDICAL RESEARCH AND INNOVATION AT KOBE, Hyogo (JP)

(72) Inventors: Koichi MORIMOTO, Wakayama (JP); Saori KUNII, Wakayama (JP); Kenji KANEKIYO, Osaka (JP); Norihiko NAKANO, Osaka (JP); Chizuka IDE, Kyoto (JP); Kaoru OMAE, Hyogo (JP)

(73) Assignees: KINKI UNIVERSITY, OSAKA (JP); AINO UNIVERSITY, Osaka (JP); FOUNDATION FOR BIOMEDICAL RESEARCH AND INNOVATION AT KOBE, Hyogo (JP)

(21) Appl. No.: 16/966,232

(22) PCT Filed: Jan. 31, 2019
(86) PCT No.: PCT/JP2019/003502

§ 371 Date: Jul. 30, 2020

(30) Foreign Application Priority Data
Jan. 31, 2018 (JP) 2018-015496

(51) Int. Cl. C07K 14/78; C12N 5/0793; A61K 38/39; A61P 25/00

(52) U.S. Cl. CPC C07K 14/78; C12N 5/0619; C12N 2501/998; A61P 25/00; A61K 38/39

(57) ABSTRACT

A nerve cell culture material including LASCol has the effect of successfully maintaining survival of the nerve cell. Furthermore, a therapeutic agent for nerve damage including LASCol can allow an endogenous nerve cell to infiltrate or proliferate actively in vivo, thereby enabling neurites to extend early and bind to each other, and has an effect on nerve damage, the effect being sufficient to improve a BBB score.

### FIELD

The present invention relates to a culture material that contains a scaffold material for culturing nerve cells and a therapeutic agent for nerve damage using the same.

### BACKGROUND

Currently, there is a need to repair a damaged central nerve. To realize this, it is necessary to culture a nerve cell, maintain survival of the nerve cell in vivo, and promote neurite extension of the nerve cell in vivo, thereby reconstructing a neural circuit.

Among nerves, the central nervous system including brain and spinal cord is believed not to be repaired spontaneously when damaged. The reasons for this are that the nerve cell in the central nervous system does not easily divide and proliferate and that body's response to the damage causes formation of hard and inflexible fibrous tissue called glial scar at the damaged site and a nerve fiber cannot extend beyond the glial scar. Another example of the reasons is that factors that inhibit neurite extension (for example, Nogo, MAG, OMgp, and Sema3A) exist in the living body and the action of these factors inhibits neurite extension.

Unlike other type of cells, a nerve cell is composed of a cell body and an axon extending from the cell body, and cells accompanying these. Therefore, culturing the nerve cells requires promoting extension of the axon or the like in addition to maintaining survival of the nerve cell.

However, a serum-containing medium conventionally used for cell culture is not sufficient to promote growth of a fragile cell such as a nerve cell, a neuroblast, or a neural stem cell. Furthermore, the serum-containing medium had the following problem: the medium remarkably promotes growth of a non-nerve cell, resulting in a very high percentage of the non-nerve cells of the total cultured cells, which was disadvantageous. Thus, a culture medium for culturing nerve cells that contains at least 2 mg of a Knitz type protease inhibitor per one liter of medium has been proposed (Patent Literature 1).

Patent Literature 2 discloses a nerve regeneration guide made by shaping a composition that contains a bioabsorbable polymer such as polylactic acid and collagen into a plate, a thread-like, or a network structure. In Patent Literature 2, the nerve regeneration guide was implanted into a part where a rat sciatic nerve was excised and the nerve was taken out after a period of time, and regeneration of the sciatic nerve was confirmed visually.

Furthermore, Patent Literature 3 discloses the following: a scaffold material for implantation produced by bonding a needle-like magnetic substance to one end of a fibrous structure composed of a biodegradable polymer selected from the group consisting of polyglycolic acid, polylactic acid, and a glycolic acid/lactic acid copolymer; or a scaffold material for implantation produced by inserting the needle-like magnetic substance into the lumen of the fibrous structure composed of the biodegradable polymer.

Patent Literature 3 has demonstrated, by using a BBB (Basso, Beattie, Bresnahan) score (score for rating motor paralysis), that restoration of movement was seen in a rat with spinal cord injury when the above-mentioned scaffold structure was implanted in the rat.

As shown in the above-mentioned literatures, the nerve cell needs to extend a projection such as an axon, and therefore, culturing (including growth in the body) thereof requires a scaffold that has bioaffinity or biodegradability.

As is described in Patent Literature 2, collagen is a material that has bioaffinity and is also readily available. It is known that there are many types of collagen. Collagen has a triple helical structure composed of a chains. Patent Literature 4 describes low adhesive collagen (Low Adhesive Scaffold Collagen, hereinafter referred to as “LASCol”) that was produced by cleaving these a chains at the end thereof by using a specified enzyme. LASCol is known as a scaffold material for culturing cells (Patent Literature 4).

When a scaffold using LASCol is utilized instead of a scaffold using conventional collagen, cells to be cultured form an aggregate (spheroid), and thus, the cells to be cultured can be cultured in a three-dimensional form, which is more similar to in vivo state (Patent Literature 4). Such LASCol is also effective in promoting differentiation of stem cells (Patent Literature 5).

Patent Literature 6 discloses a therapeutic agent for central nerve injury that uses TGF-β1, which is a growth factor.

### CITATION LIST

### Patent Literature

Patent Literature 1: Japanese Patent Application Laid-Open No. Hei. 07-046982 (1995-046982)
Patent Literature 2: Japanese Patent Application Laid-Open No. 2007-177074
Patent Literature 3: Japanese Patent Application Laid-Open No. 2014-014382
Patent Literature 4: International Publication No. 2015/167003
Patent Literature 5: International Publication No. 2015/167004
Patent Literature 6: International Publication No. 2010/024432

### Non-Patent Literature

Non-Patent Literature 1: K. Morimoto et al., Bioscience, Biotechnology, and Biochemistry, Vol. 68, pp. 861-867, 2004

### SUMMARY

### Technical Problem

However, it was unknown whether the above-mentioned substance such as collagen or LASCol was effective for maintenance of survival of the nerve cell and neurite extension. A form thereof that may be easily administered to an affected part (for example, injured spinal cord) for repairing a damaged central nerve has also been unknown.

### Solution to Problem

The present inventors have found that LASCol was effective for maintenance of survival of a nerve cell and extension of an axon, thereby completing the present invention.

More specifically, a nerve cell culture material according to the present invention contains LASCol. A therapeutic agent for nerve damage according to the present invention also contains LASCol.

The present invention can provide a method for culturing nerve cells by using the above-mentioned nerve cell culture material and can also provide a method for treating nerve damage by using the above-mentioned therapeutic agent for nerve damage.

### Advantageous Effects of Invention

A component (LASCol) contained in the nerve cell culture material and the therapeutic agent for nerve damage according to the present invention is nontoxic and has a high bioaffinity.

Furthermore, when the pH of the therapeutic agent for nerve damage according to the present invention is adjusted and the temperature thereof is raised, the form of LASCol therein changes from a liquid state to a gel state. Therefore, LASCol can be injected as liquid and thus can be administered more easily to an affected part (for example, injured spinal cord), which makes treatment using LASCol less invasive. Furthermore, LASCol tends to stay in the affected part after injected into the body. Consequently, the frequency of administration required while the nerve cells grow can be reduced, which makes a patient's burden light.

What enables such administration is presumed to be the following properties of the component (LASCol) contained therein: LASCol is less viscous even at a high concentration and has a slower fiber formation rate compared with conventional collagen. These properties are presumably due to “the structure of LASCol resulting from a specified enzymatic treatment that truncates the end of an α chain (a telopeptide region likely to cause allergic reaction) while maintaining a triple helical structure.”

### BRIEF DESCRIPTION OF DRAWINGS

FIG. 1 is a graph showing change in the elastic modulus for different concentrations of LASCol solutions over time.

FIG. 2 is a graph showing relationship between strain and stress for different concentrations of LASCol.

FIG. 3 includes phase-contrast photomicrographs showing the result of culturing of a nerve cell for 48 hours, in the case of a LASCol-coated group (indicated as LASCol in the figure), an atelocollagen-coated group (indicated as Atelocollagen in the figure), poly-L-lysine-coated group (indicated as PLL in the figure), and a control group (indicated as Non-coated in the figure).

FIG. 4 is an enlarged SEM photograph of the nerve cell in the LASCol-coated group in FIG. 3.

FIG. 5 is a further enlarged SEM photograph of FIG. 4.

FIG. 6 is an enlarged SEM photograph of the nerve cell in the atelocollagen-coated group in FIG. 3.

FIG. 7 is a further enlarged SEM photograph of FIG. 6.

FIG. 8 is an enlarged SEM photograph of the nerve cell in the poly-L-lysine-coated group in FIG. 3.

FIG. 9 is a further enlarged SEM photograph of FIG. 8.

FIG. 10 is a graph showing the result of cell counting of cultured astrocytes, in the case of the LASCol-coated group (indicated as LASCol in the figure), the atelocollagen-coated group (indicated as Atelocollagen in the figure), and the control group (indicated as Non-coated in the figure).

FIG. 11 includes phase-contrast photomicrographs showing the result of culturing of a bone marrow stromal cell for 7 days, in the case of the LASCol-coated group (indicated as LASCol in the figure), the atelocollagen-coated group (indicated as Atelocollagen in the figure), the poly-L-lysine-coated group (indicated as PLL in the figure), and the control group (indicated as Non-coated in the figure).

FIG. 12 is a graph showing the result of cell counting of the cultured bone marrow stromal cells, in the case of the LASCol-coated group (indicated as LASCol in the figure) and the control group (indicated as Non-coated in the figure).

FIG. 13 includes phase-contrast photomicrographs showing the result of culturing of a macrophage for 48 hours, in the case of the LASCol-coated group (indicated as LASCol in the figure) and the atelocollagen-coated group (indicated as Atelocollagen in the figure).

FIG. 14 is a graph showing the result of assessment based on a BBB locomotor rating scale for the LASCol-receiving group (indicated as LASCol in the figure) and the control group (indicated as PBS in the figure).

FIG. 15 includes fluorescence photomicrographs showing the result of staining of the astrocyte in the injured part of a spinal cord with an anti-GFAP antibody.

FIG. 16 includes fluorescence photomicrographs showing the result of staining a regenerated nerve in the injured part of the spinal cord with anti-phosphorylated GAP-43 antibody.

FIG. 17 includes photographs showing the result of staining of the cross section of the spinal cord two weeks after implantation of an atelocollagen sponge sample.

FIG. 18 includes enlarged photographs of FIG. 17.

FIG. 19 includes photographs showing the result of staining of the cross section of the spinal cord two weeks after implantation of a LASCol sponge sample.

FIG. 20 includes enlarged photographs of FIG. 19.

FIG. 21 is a graph showing the amount of neural axons in the sponge sample calculated from the cross-sectional photograph.

### DESCRIPTION OF EMBODIMENTS

Hereinafter, a nerve cell culture material and a therapeutic agent for nerve damage according to the present invention will be described with reference to figures and Examples.

The following description is merely illustrative of an embodiment and an example of the present invention and the present invention is not limited to the following description. The following description may be modified without departing from the spirit of the invention.

LASCol that is used as a material for the nerve cell culture material and the therapeutic agent for nerve damage according to the present invention contains a degradation product of collagen or atelocollagen. Alternatively, LASCol may be used alone. Adhesiveness of collagen to cells has been weakened in the degradation product, and thus, the degradation product has the property of becoming low adhesive.

LASCol can be obtained by degrading collagen or atelocollagen enzymatically. The peptide sequence of LASCol varies depending on a degradation condition. In other words, a different type of LASCol can be obtained by using a different degradation condition.

The characteristic of LASCol that can be used in the present invention is that LASCol consists of a combination of a chains in which a chemical bond between Y₁ and Y₂ is cleaved in an amino-terminal amino acid sequence including a triple helical domain of collagen or atelocollagen, the sequence being shown below (A).

(SEQ ID NO: 1)
(A)-Y₁-Y₂-Y₃-G-Y₄-Y₅-G-Y₆-Y₇-G-Y₈-Y₉-G-

(where G represents glycine, and Y₁ to Y₉ each represent an optional amino acid)

The triple helical domain of collagen is known to have a succession of -G-X—Y— sequences (where G represents glycine, and X and Y each represent an optional amino acid). In the above-mentioned sequence, “G” in “—Y₃-G-Y₄-Y₅-” represents glycine on the N-terminal side of the triple helical domain. As can be seen from the above-mentioned sequence, the cleavage of the chemical bond between Y₁ and Y₂ is cleavage that was carried out outside of the triple helical domain. As described below, a different degradation condition leads to cleavage inside of the triple helical domain. One of the LASCols used in the present invention is LASCol in which cleavage has occurred outside of the triple helical domain. Hereinafter, this LASCol is referred to as LASCol-A.

LASCol, which is used in the nerve cell culture material and the therapeutic agent for nerve damage according to the present invention, can be favorably used particularly for maintenance of survival of a nerve cell or neurite extension. As shown in Examples described below, LASCol-A has a very poor ability to culture a cell other than the nerve cell. However, LASCol-A has an ability to maintain survival of the nerve cell and promote extension of a nerve fiber.

It is known that the following LASCol is obtained under a certain degradation condition. Such LASCol consists of a combination of α chains in which a chemical bond between X₁ and X₂, a chemical bond between X₂ and G, a chemical bond between G and X₃, a chemical bond between X₄ and G, or a chemical bond between X₆ and G is cleaved in an amino-terminal amino acid sequence including a triple helical domain of collagen or atelocollagen, the sequence being shown below (B).

| | |
|---|---|
| (B)-G-X₁-X₂-G-X₃-X₄-G-X₅-X₆-G- | (SEQ ID NO: 2) |

(where G represents glycine, and X₁ to X₆ each represent an optional amino acid)

This is referred to as LASCol-B. In LASCol-B, cleavage has occurred inside of the triple helical domain. In SEQ ID NO: 2, G in “-G-X₁—X₂-G-” is glycine on the N-terminal side of the triple helical domain. Needless to say, there may be other LASCols that contain other peptides. Among currently known LASCols, LASCol-A is most favorable from the viewpoint of maintenance of survival of the nerve cell and occurrence of neurite extension. However, other LASCols are not excluded.

Furthermore, the nerve cell culture material and the therapeutic agent for nerve damage may contain a growth factor for the nerve cell.

The LASCol used for the nerve cell culture material and the therapeutic agent for nerve damage according to the present invention can be stored as a solution under an acidic condition. The LASCol turns into a gel state when pH and a concentration thereof are adjusted and a temperature thereof is raised to body temperature. Gelling suppresses diffusion of LASCol in the body, and LASCol exerts the effect of culturing the nerve cells in the affected part for a long period of time. In the present invention, culturing of nerve cells also includes, for example, allowing the nerve cell to survive in a form close to that in vivo (the nerve cell can survive well) and to extend an axon (neurite) thereof.

The elastic modulus of gelled LASCol is proportional to the concentration of LASCol in the solution, pH, and temperature. In Examples described below, an embodiment is illustrated in which the pH and concentration of LASCol are adjusted to prepare liquid LASCol, and the liquid LASCol is sucked into a syringe and administered by injection into the affected part, thereby allowing the LASCol to turn into gel in the affected part. However, the LASCol used as the nerve cell culture material and the therapeutic agent for nerve damage according to the present invention may be shaped into a film form or a sponge form and be implanted in the affected part. In this context, the film form or the sponge form refers to LASCol that was processed into a specified shape (also referred to as a shaped form).

As described below, it can be stated that the LASCol used in the present invention turns into gel when a concentration thereof is 3.5 mg/ml (20 Pa in terms of “practical elastic modulus” described below) or more. Therefore, when the concentration of the LASCol used as the nerve cell culture material and the therapeutic agent for nerve damage is 3.5 mg/ml or higher, the LASCol can stay in the body and regenerate the nerve cell when administered into the body.

Findings about a method for producing LASCol are almost the same for both LASCol-B and LASCol-A. Thus, findings common to both are described simply as findings about LASCol. In the following description, “degradation product” means LASCol.

### <Material for LASCol>

Collagen or atelocollagen as a material for LASCol is not limited to any particular one and may be any well-known collagen or atelocollagen.

Examples of the collagen include collagens of mammals (for example, a cow, a pig, a rabbit, a human, a rat, or a mouse), birds (for example, a chicken), fishes (for example, a shark, a carp, an eel, a tuna [for example, a yellowfin tuna], a tilapia, a sea bream, or a salmon), or reptiles (for example, a soft-shelled turtle).

Examples of the collagen used in the present invention include collagen derived from, for example, a dermis, a tendon, a bone, or a fascia of any of the above-mentioned mammals or the above-mentioned birds, collagen derived from, for example, a skin or a scale of any of the above-mentioned fishes, and collagen derived from, for example, a dermis, a tendon, or a bone of any of the above-mentioned reptiles.

Examples of the atelocollagen used for producing LASCol include atelocollagen that is produced by treating collagen of any of the above-mentioned mammals, birds, fishes, or reptiles with a protease (for example, pepsin), wherein a telopeptide has been partially removed from the amino terminus and/or the carboxyl terminus of the collagen molecule.

Among the above examples, collagen or atelocollagen of a chicken, a pig, a cow, a human, or a rat can be preferably used. More preferably, collagen or atelocollagen of a pig, a cow, or a human can be used as the material for LASCol.

Furthermore, the collagen or atelocollagen of a fish can be used as the material for LASCol. Using a fish allows for obtaining the material easily and safely in a large quantity and providing a degradation product of collagen or atelocollagen (LASCol) that is virus-free and safer to humans.

When collagen or atelocollagen of a fish is used as the material for LASCol, it is preferable to use collagen or atelocollagen of a shark, a carp, an eel, a tuna (for example, a yellowfin tuna), a tilapia, a sea bream, or a salmon; and it is more preferable to use collagen or atelocollagen of a tuna, a tilapia, a sea bream, or a salmon.

When atelocollagen is used as the material for LASCol, it is preferable to use atelocollagen that has a heat denaturation temperature of preferably 15° C. or higher, and more preferably 20° C. or higher. For example, when the atelocollagen of a fish is used as the material for the degradation product, it is preferable to use the atelocollagen of a tuna (for example, a yellowfin tuna), a tilapia, a carp, or the like, since such atelocollagen has a heat denaturation temperature of not lower than 25° C.

The above-mentioned arrangement allows for adjusting a denaturation temperature (temperature at which a substance turns into gel) of the nerve cell culture material and the therapeutic agent for nerve damage of this embodiment preferably to 15° C. or higher, and more preferably to 20° C. or higher. Consequently, the above-mentioned arrangement allows for providing a nerve cell culture material and a therapeutic agent for nerve damage that are excellent in stability during storage and stability during use.

Such collagen or atelocollagen may be obtained by a well-known method. For example, collagen-rich tissue of a mammal, a bird, or a fish may be put into an acidic solution with a pH of about 2 to 4, thereby eluting collagen. Furthermore, a protease such as pepsin is added to the eluate to partially remove a telopeptide at the amino terminus and/or carboxyl terminus of the collagen molecule. Furthermore, a salt such as sodium chloride may be added to the eluate to precipitate atelocollagen.

LASCol is obtained by allowing an enzyme to act on collagen or atelocollagen, thereby degrading such material. Alternatively, LASCol can also be obtained by producing a degradation product of collagen or atelocollagen (for example, by chemical synthesis or expression of recombinant protein), wherein the degradation product has an already cleaved chemical bond within the triple helical domain.

Hereinafter, a method for obtaining LASCol by degrading the above-mentioned collagen or atelocollagen with an enzyme (for example, protease) will be described.

The enzyme is not limited to any particular one. For example, a cysteine protease is preferably used.

It is preferable to use, as the cysteine protease, a cysteine protease that contains a larger amount of basic amino acids than the amount of acidic amino acids, or a cysteine protease that is active at a hydrogen ion concentration in the acidic range.

Examples of such a cysteine protease may include actinidain [EC 3.4.22.14], papain [EC 3.4.22.2], ficin [EC 3.4.22.3], bromelain [EC 3.4.22.32], cathepsin B [EC 3.4.22.1], cathepsin L [EC 3.4.22.15], cathepsin S [EC 3.4.22.27], cathepsin K [EC 3.4.22.38], cathepsin H [EC 3.4.22.16], alloline, and a calcium dependent protease. The text in square brackets represents an enzyme code number.

Among these, it is preferable to use actinidain, papain, ficin, cathepsin K, alloline, or bromelain, and it is more preferable to use actinidain, papain, ficin, or cathepsin K.

The above-mentioned enzyme can be obtained by a known method. For example, the enzyme can be obtained by producing the enzyme by chemical synthesis; extracting the enzyme from a cell or tissue of a bacterium, a fungus, or various animals and plants; producing the enzyme by a genetic engineering process; or other methods. Needless to say, a commercially available enzyme can also be used.

When collagen or atelocollagen is cleaved by degrading the same with an enzyme (for example, a protease), the cleaving step can be carried out by, for example, any of the methods (i) to (iii) described below. The following methods (i) to (iii) are merely examples of the cleaving step, and the method for producing LASCol is not limited to these methods (i) to (iii).

LASCol-B can be obtained by the following methods (i) and (ii). LASCol-A and LASCol-B can be obtained by the following method (iii).

(i) A method that includes bringing collagen or atelocollagen into contact with an enzyme in the presence of a high concentration of salt.
(ii) A method that includes bringing collagen or atelocollagen into contact with an enzyme that has been in contact with a high concentration of salt.
(iii) A method that includes bringing collagen or atelocollagen into contact with an enzyme in the presence of a low concentration of salt.

Specific examples of the above-mentioned method (i) may include a method that includes bringing collagen or atelocollagen into contact with an enzyme in an aqueous solution containing a high concentration of salt.

Specific examples of the above-mentioned method (ii) may include a method that includes bringing an enzyme into contact with an aqueous solution containing a high concentration of salt in advance and subsequently bringing collagen or atelocollagen into contact with the enzyme.

Specific examples of the above-mentioned method (iii) may include a method that includes bringing collagen or atelocollagen into contact with an enzyme in an aqueous solution containing a low concentration of salt. The specific composition of the above-mentioned aqueous solution is not particularly limited. For example, water can be used.

Although the specific composition of the above-mentioned salt is not particularly limited, a chloride is preferably used. The chloride is not limited to any particular one. For example, NaCl, KCl, LiCl, or MgCl₂ can be used.

Although the concentration of the salt in the above-mentioned aqueous solution containing a high concentration of salt is not particularly limited, a higher concentration is more preferable. For example, the concentration is preferably 200 mM or higher, more preferably 500 mM or higher, still more preferably 1000 mM or higher, even more preferably 1500 mM or higher, and most preferably 2000 mM or higher.

Although the concentration of the salt in the above-mentioned aqueous solution containing a low concentration of salt is not particularly limited, a lower concentration is more preferable. For example, the concentration is preferably 200 mM or lower, more preferably 150 mM or lower, still more preferably 100 mM or lower, even more preferably 50 mM or lower, and most preferably substantially 0 mM.

Although collagen or atelocollagen may be dissolved in the above-mentioned aqueous solution (for example, water) in any amount, by way of example, it is preferable that 1 part by weight of collagen or atelocollagen be dissolved in 1000 parts by weight to 10000 parts by weight of the aqueous solution.

The above-mentioned arrangement enables efficient contact between an enzyme and the collagen or atelocollagen when the enzyme is added to the aqueous solution. Consequently, the collagen or atelocollagen can be degraded efficiently with the enzyme.

Although the enzyme may be added to the aqueous solution in any amount, by way of example, it is preferable that 10 parts by weight to 20 parts by weight of the enzyme be added to 100 parts by weight of the collagen or atelocollagen.

The above-mentioned arrangement, in which the concentration of the enzyme in the aqueous solution is high, enables efficient degradation of the collagen or atelocollagen with the enzyme (for example, a protease).

Furthermore, other conditions (for example, the pH of the aqueous solution, temperature, and a contact time) under which the collagen or atelocollagen is brought into contact with the enzyme in the aqueous solution are not particularly limited and may be selected as appropriate. However, these conditions are preferably within the ranges described below. Preferable ranges of these conditions are illustrated below.

1) The pH of the aqueous solution is preferably 2.0 to 7.0, and more preferably 3.0 to 6.5. For keeping the pH of the aqueous solution within the above-mentioned range, a well-known buffer may be added to the aqueous solution. The above-mentioned pH allows the collagen or atelocollagen to be dissolved in the aqueous solution uniformly, and consequently allows the enzymatic reaction to proceed efficiently.

2) The temperature is not limited to any particular value and may be selected depending on the enzyme to be used. The temperature is, for example, preferably 15° C. to 40° C., and more preferably 20° C. to 35° C.

3) The contact time is not limited to any particular length and may be selected depending on the amount of the enzyme and/or the amount of the collagen or atelocollagen. The contact time is, for example, preferably 1 hour to 60 days, more preferably 1 day to 7 days, and even more preferably 3 days to 7 days.

When necessary, at least one step selected from the group consisting of a step of readjusting the pH, a step of inactivating the enzyme, and a step of removing contaminants may be performed after allowing the collagen or atelocollagen to be in contact with the enzyme in the aqueous solution.

The step of removing contaminants can be carried out by a general method for separating a substance. The step of removing contaminants can be carried out by, for example, dialysis, salting-out, gel filtration chromatography, isoelectric precipitation, ion exchange chromatography, or hydrophobic interaction chromatography.

The nerve cell culture material according to the present invention is used, for example, as follows: firstly a solution containing LASCol is coated onto a culture dish, secondly a culture medium such as D-MEM (Dulbecco's modified Eagle's medium) is added onto the culture dish, and then the nerve cells are seeded thereon.

The therapeutic agent for nerve damage according to the present invention is administered to the affected part after identifying the damaged area after a certain period of time has passed since the nerve was damaged. For example, in the case of spinal cord, the therapeutic agent for nerve damage is administered to the affected part, for example, by injection after the injured part of the spinal cord was identified, for example, by roentgenography not immediately after spinal cord injury but after a certain period of time has passed. In this case, it is desirable that the LASCol contained in the therapeutic agent for nerve damage have an elastic modulus (“practical elastic modulus” described below) not less than a predetermined value. This is because there is a risk that LASCol with a low elastic modulus may not stay in the affected part and flow out therefrom.

The nerve cell culture material or the therapeutic agent for nerve damage according to the present invention is provided, for example, in a dry state (including powder and a shaped form) or a gel state. The expression “using the nerve cell culture material or the therapeutic agent for nerve damage according to the present invention at a predetermined concentration” includes a case where instructions to add a certain amount of solvent to LASCol in a dry state are attached to the product or passed on to the user, and in accordance with the instructions, a favorable concentration of LASCol of the present invention is prepared.

“Administration” as used herein means administering a therapeutic agent to a patient via the affected part. Thus, administration of the therapeutic agent according to the present invention includes not only injection but also insertion of the therapeutic agent into a site incised by incision, application of the therapeutic agent onto the affected part, and the like. Furthermore, the therapeutic agent for nerve damage according to the present invention can be regarded as a method for treating nerve damage by using the therapeutic agent for nerve damage according to the present invention.

Nerve damage to be treated by using the present invention is damage in the central nerve area and the peripheral nerve area; and examples thereof include traumatic injury caused by an accident such as traffic accident, sport accident, and a fall, and damage caused by a disease such as spinal cord tumor and hernia.

### EXAMPLES

### <Preparation of Solution Containing LASCol>

50 mM citric acid buffer solutions (pH 3.0) each containing sodium chloride at a concentration of 0 mM or 1500 mM were prepared. Water was used as a solvent of these aqueous solutions.

For activating actinidain, actinidain was dissolved in 50 mM phosphate buffer (pH 6.5) containing 10 mM dithiothreitol and the resultant aqueous solution was left to stand at 25° C. for 90 minutes. Note that actinidain had been purified by a well-known method before use (see, for example, Non-Patent Literature 1).

Next, pig-derived type I collagen was dissolved in the 50 mM citric acid buffer solution containing the salt (pH 3.0). The resultant solution containing the pig-derived type I collagen was brought into contact with the aqueous solution containing actinidain at 20° C. for 10 days or longer to produce a degradation product of type I collagen. Note that the pig-derived type I collagen had been purified by a well-known method (see, for example, Non Patent Literature 1).

The above-mentioned degradation product was subjected to sodium lauryl sulfate-polyacrylamide gel electrophoresis (SDS-PAGE) to separate the degradation product of type I collagen.

Subsequently, the degradation product of type I collagen was transferred onto a PVDF (polyvinylidene difluoride) membrane by a routine method. Then, an amino acid sequence of the amino terminus of a degradation product of an α1 chain transferred onto the PVDF membrane was determined by the Edman degradation technique.

Note that APRO Science Inc. or Collaborative Laboratory (Analytical tools) of the Faculty of Medicine of Kindai University conducted the actual Edman analysis in accordance with a well-known method, at the request of the present inventors.

Table 1 shows the amino acid sequence of the amino terminus and the vicinity thereof of the degradation products of the α1 chain that were obtained at salt concentrations of 0 mM and 1500 mM.

As shown in Table 1, cleavage occurred outside of a triple helical domain represented by “GPMGPSGPRG⋅ ⋅ ⋅” when the salt concentration was low (0 mM), while cleavage occurred inside of the triple helical domain when the salt concentration was high (1500 mM). In SEQ ID NO: 3, the triple helical domain starts from glycine (G) that is the third amino acid from the left. A solution produced in the case of 0 mM is a LASCol-A solution and a solution produced in the case of 1500 mM is a LASCol-B solution. In the following Examples, the LASCol-A solution was used as the LASCol solution.

TABLE 1

| SALT | | |
|---|---|---|
| CON- | | |
| CEN- | | |
| TRA- | AMINO-TERMINAL SEQUENCE OF | SE- |
| TION | DEGRADATION PRODUCT OF PIG- | QUENCE |
| [mM] | DERIVED α1 CHAIN | NUMBER |
| 0 | V P G P M G P S G P R G . . . | 3 |
| 1500 | M G P S G P R G . . . | 4 |

In LASCol-A, cleavage also occurs in an a2 chain. In Table 2, SEQ ID NO: 5 represents the amino acid-terminal portion of the α2 chain. In SEQ ID NO: 5, the triple helical domain starts from glycine (G) located at the left end of “⋅ ⋅ ⋅GPMGLMG⋅ ⋅ ⋅.” SEQ ID NO: 6 represents the end of the α2 chain produced at a salt concentration of 0 mM, which is a condition for production of LASCol-A. When compared with SEQ ID NO: 2, SEQ ID NO: 6 corresponds to a sequence resulting from cleavage of a chemical bond between G and X₃ in SEQ ID NO: 2.

In other words, in LASCol-A, cleavage in the α1 chain has occurred outside of the triple helical domain, while cleavage in the α2 chain has occurred inside of the triple helical domain. LASCol-A only needs to have either one of cleavages shown in SEQ ID NO: 3 and SEQ ID NO: 6.

TABLE 2

| SALT | | |
|---|---|---|
| CON- | | |
| CEN- | | |
| TRA- | AMINO-TERMINAL SEQUENCE OF | SE- |
| TION | DEGRADATION PRODUCT OF PIG- | QUENCE |
| [mM] | DERIVED α2 CHAIN | NUMBER |
| — | . . . G P G P M G L M G P R | 5 |
| | G P P . . . | |
| 0 | L M G P R | 6 |
| | G P P . . . | |

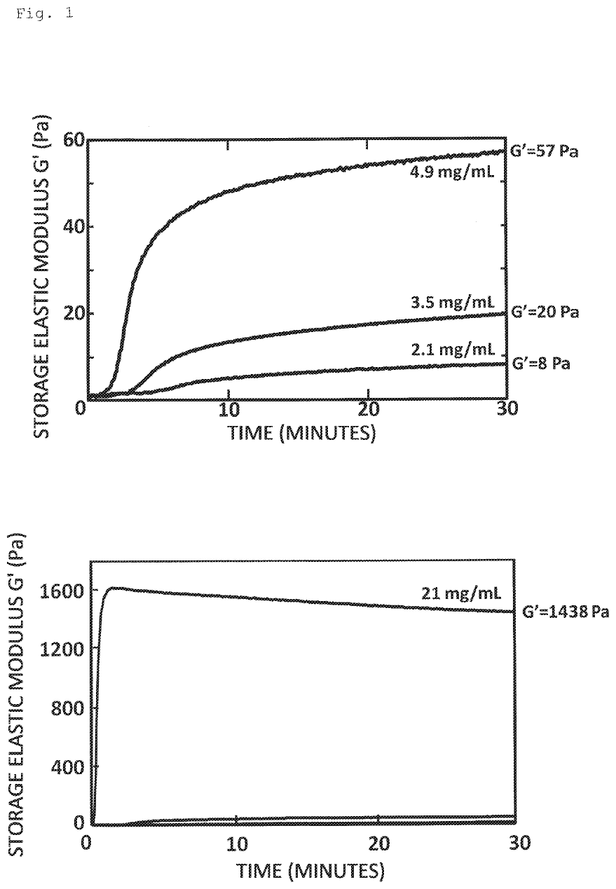

FIG. 1 shows an elastic property of a solution containing LASCol (a storage elastic modulus part G′ of complex elastic modulus). The horizontal axis represents time (minutes) and the vertical axis represents storage elastic modulus G′ (Pa). FIG. 1(a) and FIG. 1(b) have the same horizontal axes but different vertical axes. The scale of the vertical axis in FIG. 1(b) is larger than that of FIG. 1(a). Each curve in FIG. 1(a) and FIG. 1(b) corresponds to the storage elastic moduli of different concentrations of LASCol. LASCol solutions of different concentrations were prepared by using 5 mM hydrochloric acid solution so that the final LASCol concentrations became 2.1 mg/mL, 3.5 mg/mL, and 4.9 mg/mL (FIG. 1(a)), and 21 mg/ml (FIG. 1(b)).

These LASCols are stored in an acidic solution in a temperature range from 5° C. to 10° C. Under this condition, LASCol can be stored in a liquid state. FIG. 1 shows the measurement results of LASCol. For this measurement, a pH adjuster and a concentration adjusting solution were added to LASCol to adjust pH thereof to nearly 7.4, then the LASCol sample was placed in a dynamic viscoelasticity measuring device (rheometer: HAAKE MARS III, Thermo Fisher Scientific Inc.), and the temperature was raised to 37° C. before measurement. The measurement conditions were a frequency of 1 Hz, an amplitude of 6°/second, and a strain percentage of 1%. Raising temperature is completed in a few seconds.

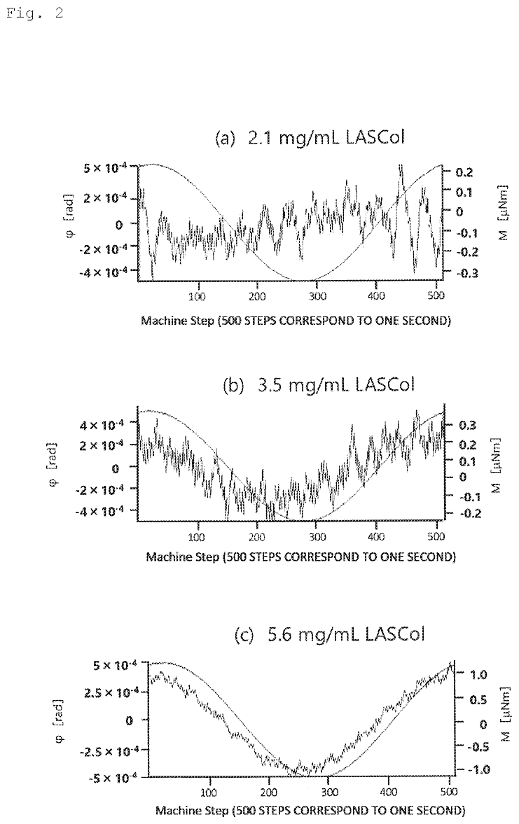

Referring to FIG. 1(a), a storage elastic modulus G′ determined immediately after the start of measurement was low regardless of the LASCol concentration. Subsequently, regardless of the LASCol concentration, the storage elastic modulus G′ increased and approached a saturation point in about 10 minutes. On the other hand, in FIG. 1(b), a storage elastic modulus G′ increased to the saturation point in 1 minute after the start of measurement, and then gradually decreased to saturation level. As is clear from FIG. 1 and FIG. 2, increasing the LASCol concentration shortened the time for the storage elastic modulus G′ to increase.

This indicated that the storage elastic modulus G′ of the solution containing LASCol increased to a certain value that depended on the LASCol concentration when the pH and concentration of the LASCol solution were adjusted and the temperature thereof was raised. Furthermore, it was found that the storage elastic modulus reached an almost stable value 30 minutes after the LASCol solution was prepared so as to have a predefined concentration and the temperature thereof was raised to 37° C. For this reason, the storage elastic modulus at this time point is referred to as “practical elastic modulus” of LASCol.

It was shown that, when LASCol was exposed to an appropriate condition, the property thereof changed from sol having an unmeasurable elastic modulus to gel having a quantifiable elastic modulus, and thus LASCol could be used as an injectable gel particularly for injection into a living body.

FIG. 2 represents the relationship between “strain (displacement in a rotation direction of a driving unit of the rheometer)” and “stress (stress received by a receiving unit of the rheometer)” after the LASCol sample was kept in the rheometer for 30 minutes at 37° C. The left vertical axis represents strain φ (rad) and the right vertical axis represents stress M (μNm). The horizontal axis represents the number of machine steps and is unitless, wherein 500 steps correspond to one second. Thus, the figures in FIG. 2 show the results of measurement during which strain p was changed from 5×10⁻⁴ rad to −5×10⁻⁴ rad and back again over a period of one second.

FIG. 2(a) represents a case where the LASCol concentration was 2.1 mg/ml, FIG. 2(b) represents a case where the LASCol concentration was 3.5 mg/ml, and FIG. 2(c) represents a case where the LASCol concentration was 5.6 mg/ml. Respective practical elastic moduli were 8 Pa, 20 Pa, and 70 Pa. When the LASCol concentration was 2.1 mg/ml (FIG. 2(a)), little response of stress to strain was observed. Thus, LASCol can be considered to be nearly liquid. When the LASCol concentration was increased to 3.5 mg/ml (FIG. 2(b)), response of stress corresponding to strain was observed.

When the LASCol concentration was further increased (FIG. 2(c)), stress came to synchronize with the applied strain. The reason why the strain and the stress are out of phase is that gel has a loss elastic modulus. Therefore, the present inventors were able to conclude that LASCol turned into gel at a LASCol concentration of 3.5 mg/ml as shown in FIG. 2(b). This concentration was equivalent to a practical elastic modulus of 20 Pa.

When LASCol is used for the therapeutic agent for nerve damage, the lower limit of the storage elastic modulus thereof in a gel form is believed to be 20 Pa. LASCol also functions as a scaffold for cells, and thus needs to stay in one place to some extent. The reason why the lower limit is 20 Pa is that LASCol with an elastic modulus of less than 20 Pa does not behave as gel and thus is believed to have difficulty in staying in the affected part.

### <Nerve Cell Culture>

The LASCol solution prepared as described above was used to confirm an ability thereof to maintain survival of the nerve cell. LASCol, atelocollagen, and poly-L-lysine (also referred to as “PLL” hereinafter) were coated onto a 24-well microplate. A non-coated well having no coating thereon was prepared as a control. Nerve cells derived from a neonatal rat hippocampus (a nerve cell that is not a mesenchymal stem cell and has completed differentiation; hereinafter simply referred to as “nerve cell”) were suspended in Neurobasal medium with B-27 supplement (manufactured by Thermo Fisher Scientific Inc., hereinafter referred to as “NB/B27”) and seeded in the above-mentioned wells.

PLL promotes adhesion mediated by an electric charge between a cell membrane surface and the culture dish. Therefore, use of PLL can make nerve cells adhere to a commercially available plastic plate, although a hydrophilicity-enhancing treatment generally applied to such a plate does not provide, by itself, sufficient adhesiveness. PLL is commonly used in culturing nerve cells.

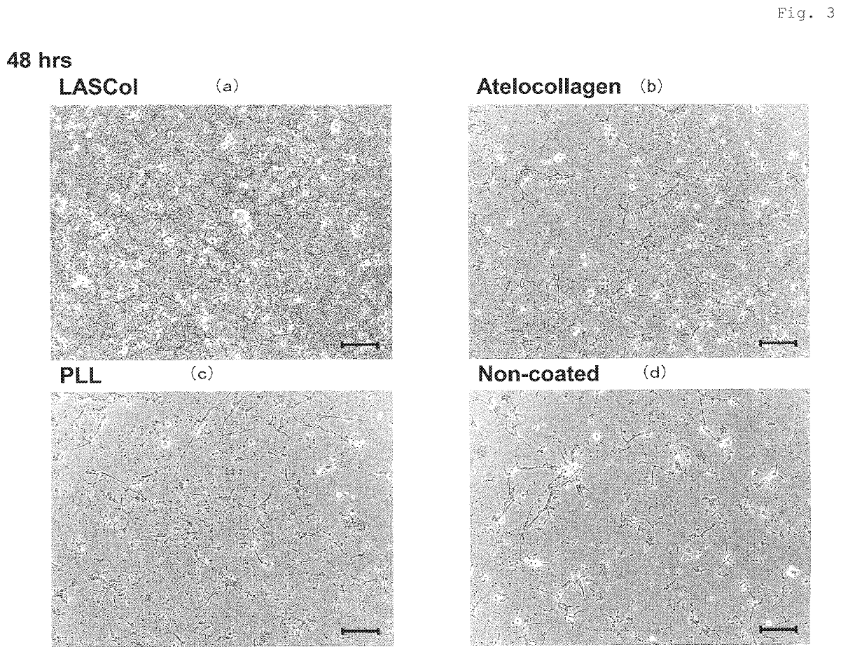

The state of the nerve cells was observed by microscopy after 48 hours of culture and the result is shown in FIG. 3. The scale bar at the bottom right in the photographs represents 100 μm. FIG. 3(a), FIG. 3(b), FIG. 3(c), and FIG. 3(d) show the results for the plate coated with the LASCol solution (LASCol-coated group), the plate coated with the atelocollagen solution (atelocollagen-coated group, indicated as “Atelocollagen”), the plate coated with the PLL solution (PLL-coated group), and the plate having no coating thereon (Non-coated group), respectively.

In FIG. 3(a), round objects are densely present and long thread-like objects extend therebetween. The round object is a cell body of the nerve cell, and the long thread-like object is a projection (neurite) that extends from the nerve cell.

In FIG. 3 (b), many cell bodies of the nerve cells are seen, but not as many as in FIG. 3 (a). Then, in the order of FIG. 3(c) and FIG. 3(d), the number of cell bodies of the nerve cells decreases. It was also confirmed that the number and length of the neurites that extended from the nerve cells were reduced in the order of FIG. 3(a) to FIG. 3 (d). It was found from the above that the nerve cells could survive well and extend the neurites thereof on LASCol.

Next, the LASCol solution, the atelocollagen solution, and the PLL solution were coated onto 20 mm×20 mm slide glasses and the nerve cells were seeded thereon. Then, the nerve cells were observed by scanning electron microscopy (SEM) after 24 hours. Specifically, the culture sample was fixed with 4% paraformaldehyde, dehydrated in alcohol, immersed in isoamyl acetate, and dried by critical point drying using liquefied carbon dioxide. Subsequently, the sample was coated with platinum palladium and observed by Hitachi S5000 SEM.

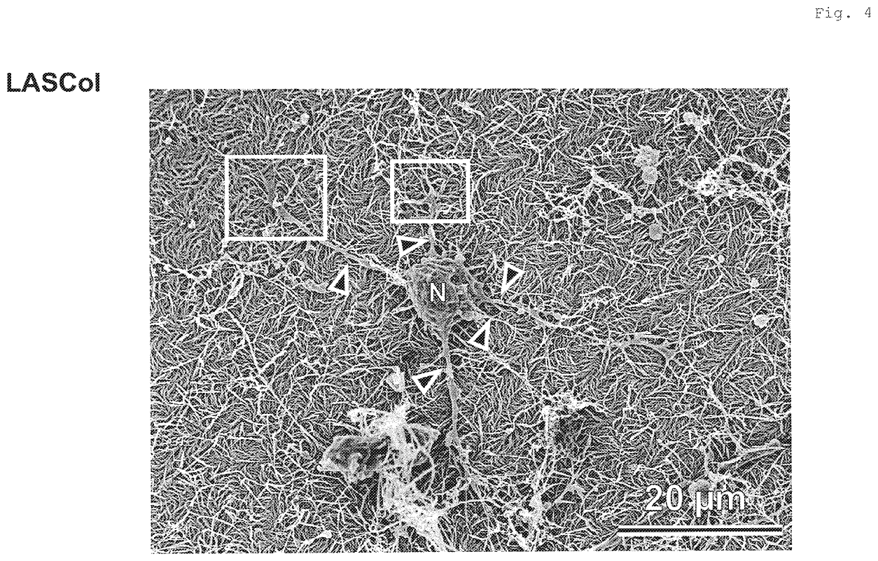

FIG. 4 shows the image of the nerve cells in the LASCol-coated group observed by SEM. The scale bar at the bottom right represents 20 μm. It was confirmed that a plurality of projections called neural axon (arrowheads in the figure) had extended from the nerve cell (a part indicated by a symbol “N” in FIG. 4) against a background of the LASCol that densely aggregated in a fibrous form. A growth cone (inside the box in the figure) that is essential for the activity of the nerve cell had formed at a place along the projection of each axon.

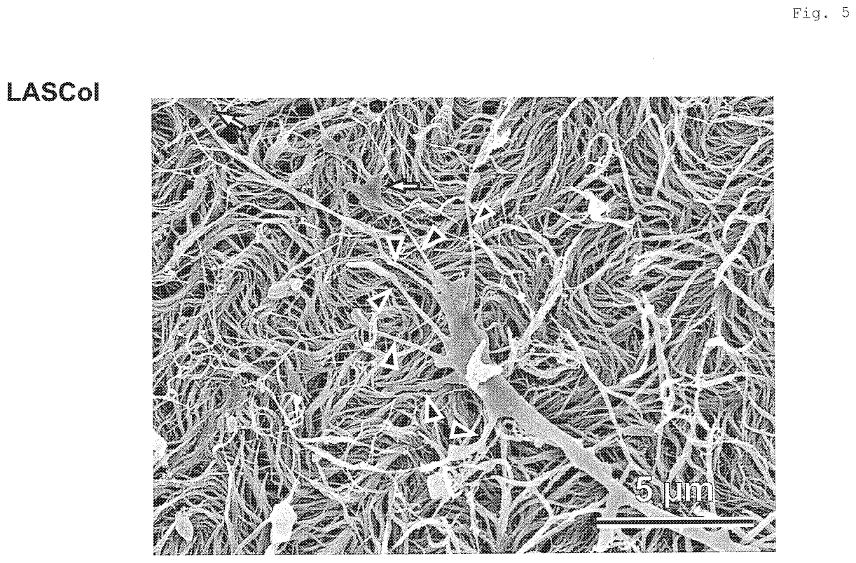

FIG. 5 is an enlarged SEM image of the growth cone in FIG. 4. The scale bar at the bottom right represents 5 μm. The growth cone is highly motile and a plurality of long and thin neurites extend therefrom to form a network with other nerve cells, which in turn leads to synapse formation.

A plurality of long thread-like pseudopodia (filopodia, indicated by arrowheads) have extended from the growth cone that formed on the LASCol, which indicated that this growth cone was very active. Additionally, a new growth cone (arrow) had formed on the axon that further extended from the filopodia. Furthermore, the projection had a regular surface and had formed a shape typical of the projection. The nerve cell whose neurite has extended in such a state may be considered to be a form of a successfully cultured nerve cell.

Furthermore, LASCol fibers had formed densely in a layer underlying the growth cone and each filopodium had adhered to the LASCol fibers distinctly. This indicates that signal from the LASCol is involved in vigorous activity of the nerve cell.

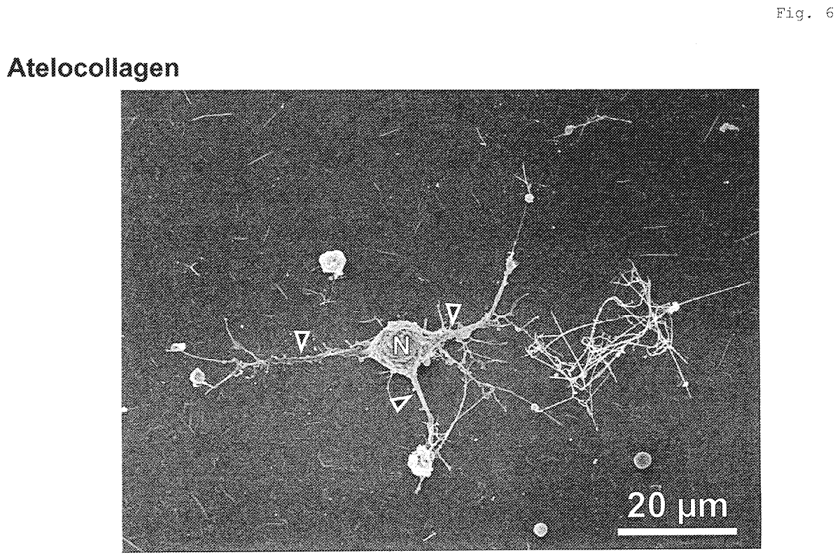

FIG. 6 shows the result of the nerve cell seeding for the atelocollagen-coated group. The scale bar at the bottom right represents 20 μm. This figure showed that although neurites (arrowhead) had extended from the nerve cell (a part indicated by a symbol “N”), the neurites were obviously less in number and shorter than those observed in the LASCol-coated group (FIG. 4).

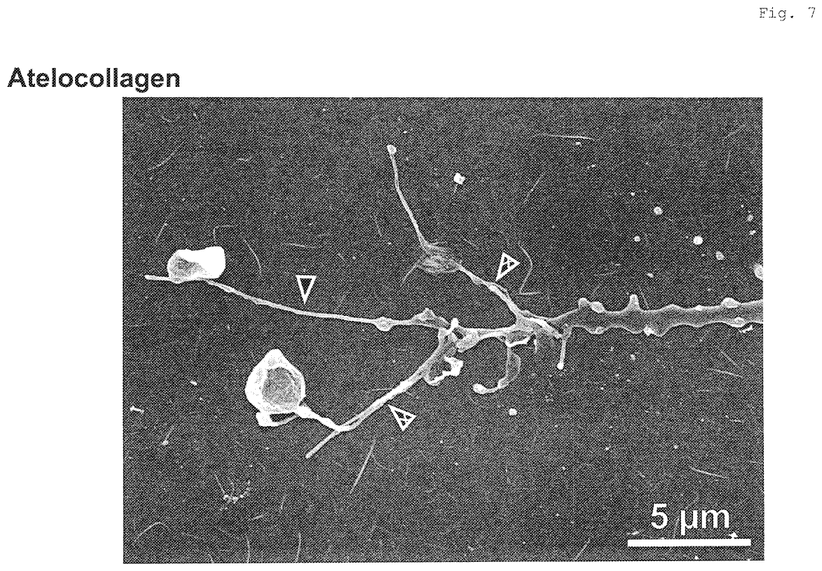

FIG. 7 is an enlarged SEM image of the growth cone in FIG. 6. The scale bar at the bottom right represents 5 μm. The growth cone that formed at the tip of the projection had changed into an irregular shape. Furthermore, filopodia (arrowhead) had not clearly formed and the nerve cell did not have an active motility. Furthermore, adhesion to the atelocollagen coated onto the layer under the nerve cells was insufficient compared with the LASCol-coated group (FIG. 5).

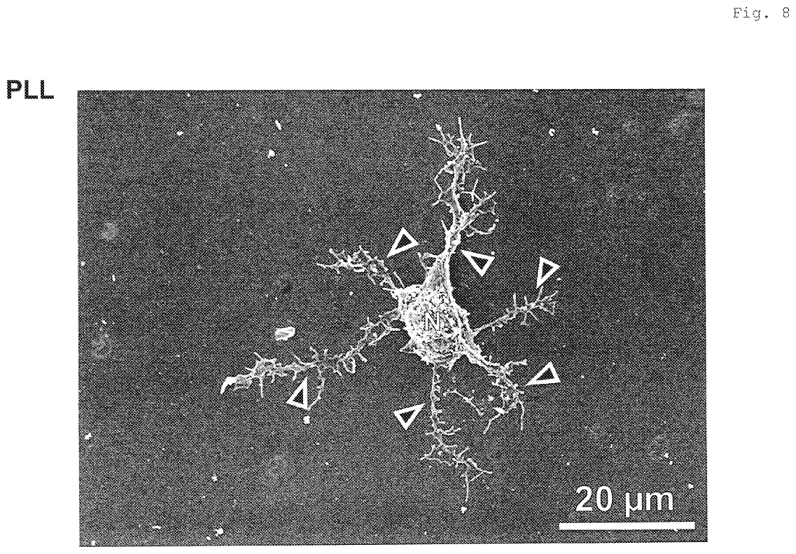

FIG. 8 shows the result for the nerve cells in the PLL-coated group. The scale bar at the bottom right represents 20 μm. Although as many as six projections had extended from the nerve cell (a part indicated by a symbol “N”), the shape of the projection was different from a common projection and showed an abnormal morphology (arrowhead). Innumerable short projections had further extended from the neurites. However, this morphology is not one observed at the projection of a normal nerve cell.

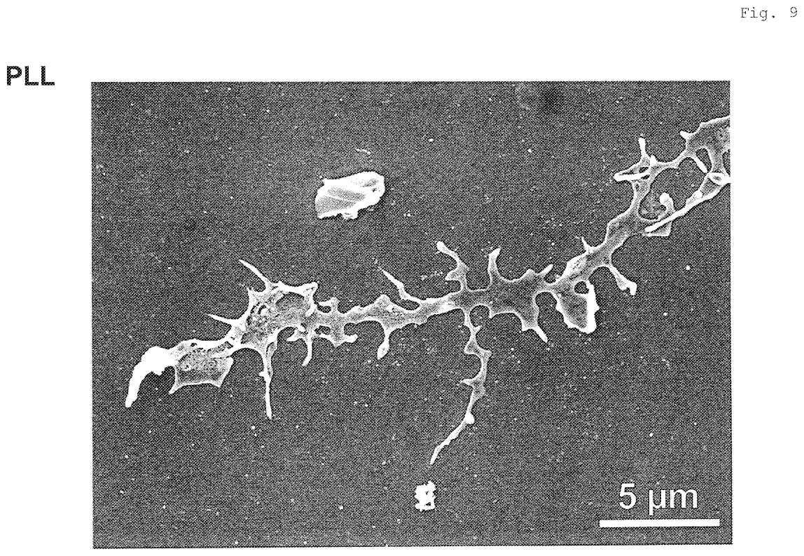

FIG. 9 is an enlarged SEM image of the growth cone in FIG. 8. The scale bar at the bottom right represents 5 μm. Formation of the growth cone was imperfect. As a whole, the nerve cell seems to be trying to extend a projection. However, extension of the projection had been suppressed, resulting in a shorter projection. The nerve cell in the PLL-coated group did not extend the projection regularly and a plurality of abnormal branched projections were observed.

It was found from the above that LASCol was effective not only for successful survival of the nerve cell but also for extension of the projection from the nerve cell.

### <Culture of Other Cells in LASCol-Coated Group>

### (1) Astrocyte

The result of culturing of an astrocyte on LASCol is shown below. LASCol and atelocollagen were coated onto a 96-well microplate. Non-coated one was also prepared as a control group. Then, astrocytes derived from a rat cerebrum were seeded at 3×10⁴ and 1×10⁵ cells/mL, and the number of cells were measured by the WST-1 method after 48 hours. The WST-1 method is one of colorimetric MMT methods. The MMT method is colorimetry by measuring the activity of an enzyme that reduces MTT or a similar dye to a formazan dye (purple).

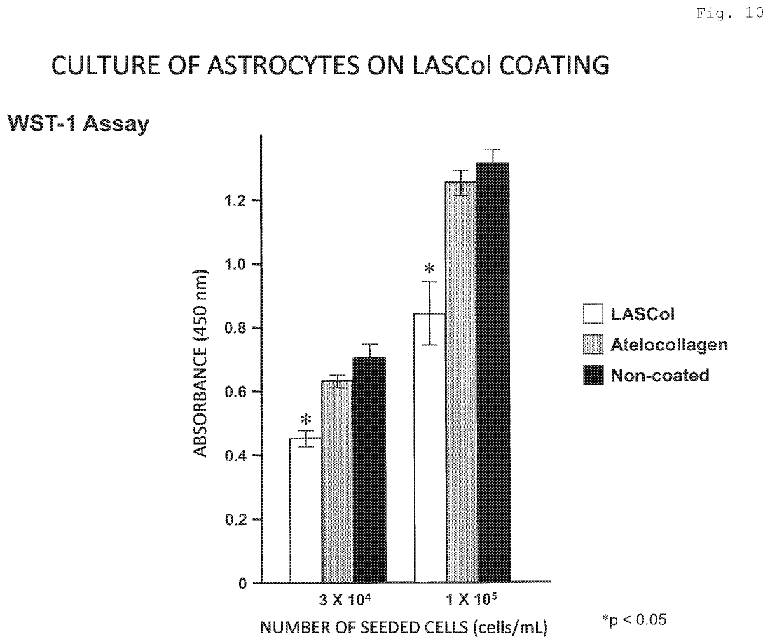

The WST-1 method is based on conversion of a tetrazolium salt (WST-1) into a formazan dye by mitochondrial dehydrogenase in a living cell and there is a linear relationship between the absorbance of the formazan dye solution and the number of living cells. Therefore, the number of cells can be quantitatively measured by measuring the absorbance. The results are shown in FIG. 10.

Referring to FIG. 10, the horizontal axis represents the number of cells for each coating material indicated for each number of seeded cells, and the vertical axis represents the absorbance at 450 nm. Regardless of the number of seeded cells, the number of cells cultured on the LASCol-coated culture dish was significantly less than the number of cells cultured on the atelocollagen-coated one and the non-coated one.

Since the astrocyte is a cell (glial cell) other than the nerve cell in the central system, the result shown in FIG. 10 indicates LASCol suppresses growth of the glial cell.

A glial cell is known to increase in a lesion in nerve tissue. When glial cells increase in a damaged area in a part with aggregated nerves, such as a spine, a nerve fiber cannot extend beyond the glial cells and thus the nerve remains severed. LASCol is believed to be able to enhance extension of the nerve fiber because LASCol suppresses growth of the glial cells.

### (2) Bone Marrow Stromal Cell

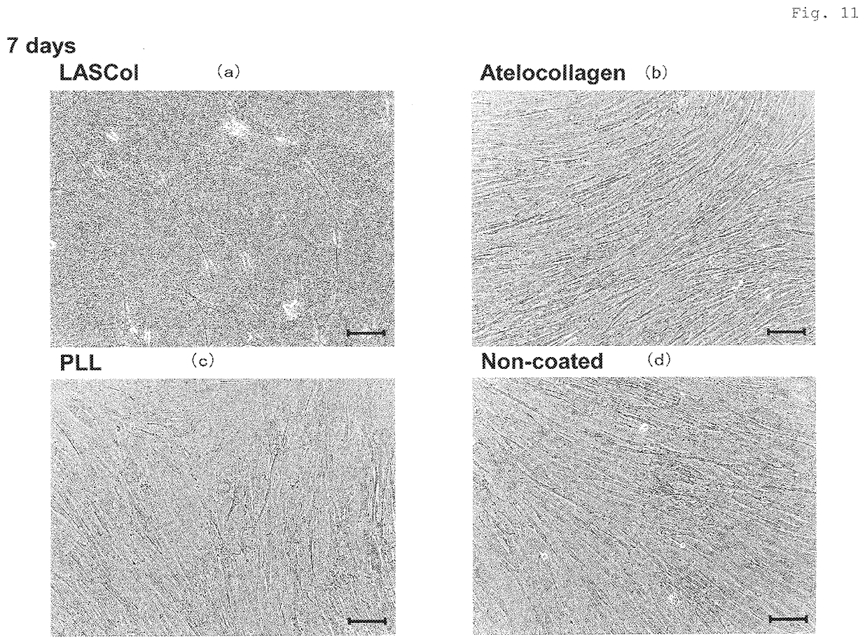

Bone marrow stromal cells were seeded at a concentration of 3×10³ cells/ml onto a dish that was coated with the LASCol solution, the atelocollagen solution, or the PLL solution and Mesenchymal Stem Cell Basal Medium with MSCGM SingleQuots (manufactured by Lonza) was added to, and observed after seven days. The results are shown in FIG. 11. FIG. 11(a), FIG. 11(b), FIG. 11(c), and FIG. 11(d) correspond to coating with the LASCol solution (LASCol-coated group), coating with the atelocollagen solution (atelocollagen-coated group), coating with the PLL solution (poly-L-lysine-coated group: PLL-coated group), and no coating (Non-coated, control group), respectively. The scale bar at the bottom right in the photographs represents 100 μm. For the LASCol-coated group in FIG. 11(a), some projections extending from a thin spindle-shaped cell body were found here and there. These are the bone marrow stromal cells. The confluency of cells was about 10%.

For the atelocollagen-coated group of FIG. 11(b), the PLL-coated group of FIG. 11(c), and the Non-coated group of FIG. 11(d), long spindle-shaped bone marrow stromal cells had adhered to each other to become confluent. It can be seen that these cells were sparse at beginning and proliferated actively to an extent that the cells fully covered the inside bottom surface.

Based on the above, one can conclude that the bone marrow stromal cells in the LASCol-coated group (FIG. 11(a)) were not able to achieve sufficient adhesion and showed less proliferation than those in the atelocollagen-coated group, the PLL-coated group, and the Non-coated group.

Furthermore, the tendency for the bone marrow stromal cells not to proliferate on LASCol was examined again. The LASCol solution was coated onto a 48-well microplate. The bone marrow stromal cells were seeded at 1×10⁵, 3×10⁴, 1×10⁴, and 3×10³ cells/well. After 24 hours, the number of cells was counted by using a Luna automated cell counter (manufactured by Logos Biosystems, Inc.). Non-coated one was also prepared as a control group.

Furthermore, the LASCol solution was coated onto a 96-well microplate and the bone marrow stromal cells were seeded at 1×10⁵, 5×10⁴, 2×10⁴, and 1×10⁴ cells/well. An assay using the WST-1 method was performed after two days.

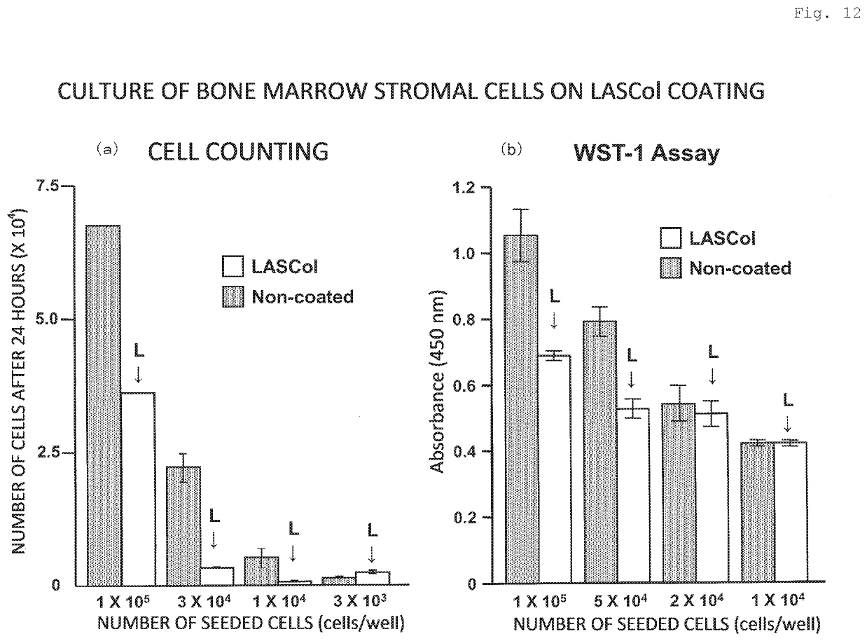

The results are shown in FIG. 12. FIG. 12(a) shows the result of cell counting. The horizontal axis represents the number of seeded cells (cells/well) and the vertical axis represents the number of cells (×10⁴) after 24 hours. When the number of seeded cells was large, culturing of the LASCol-coated group (indicated by “L” in the figure) resulted in a significant decrease in the number of cells compared with the control group (Non-coated).

FIG. 12(b) shows the result of the WST-1 method. The horizontal axis represents the number of seeded cells (cells/well) and the vertical axis represents the absorbance at 450 nm. Again, it was successfully confirmed that when the number of seeded cells was large, culturing of the LASCol-coated group (indicated by “L” in the figure) resulted in a significant decrease in the number of cells compared with the control group (Non-coated).

The above results demonstrated the tendency for the bone marrow stromal cells to proliferate less on LASCol.

### (3) Macrophage

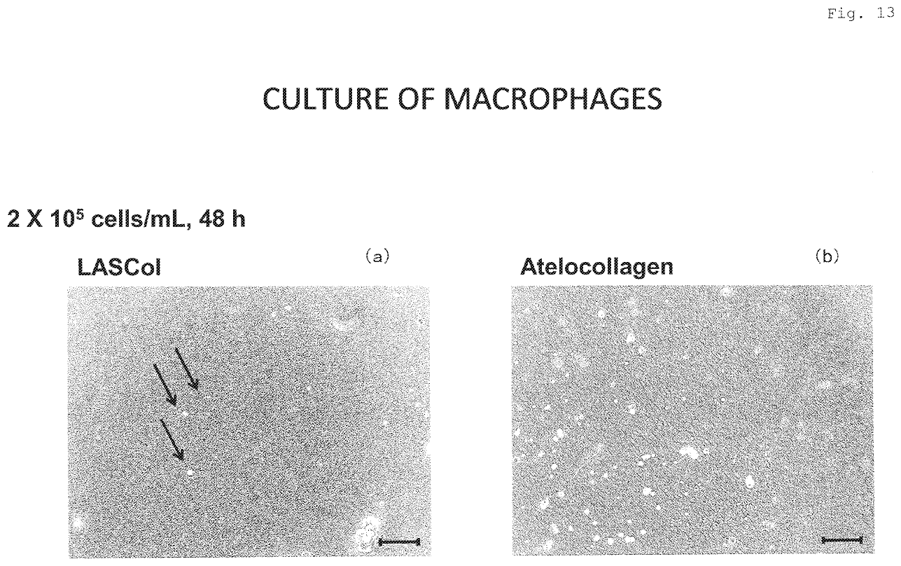

The result of culturing of macrophages on LASCol is shown below. LASCol or atelocollagen was coated onto an eight-well chamber. Then, rat peritoneal macrophages were seeded at 2×10⁵ cells/mL and observed after 48 hours. The results are shown in FIG. 13.

FIG. 13(a) shows the LASCol-coated group and FIG. 13(b) shows the atelocollagen-coated group. Each scale bar represents 100 μm. In FIG. 13(a), a spherical cell indicated by an arrow (three cells were indicated by way of illustration) is a macrophage. In FIG. 13(a), only a few macrophages were observed. On the other hand, a greater number of cells were observed clearly in FIG. 13(b) than in FIG. 13(a). Note that no arrow is shown in FIG. 13(b).

As described above, the astrocytes, the bone marrow stromal cells, and the macrophages were hardly able to proliferate on LASCol. These findings demonstrated that LASCol exerted the effect of maintaining cell survival and the effect of promoting neurite extension on the nerve cell. Therefore, it can be concluded that LASCol could be used favorably as a nerve cell culture material. Particularly, it is almost impossible to culture a non-nerve cell on LASCol, and thus the nerve cell culture material using LASCol may allow for culturing the nerve cell in a state close to an actual state even when other cells coexist.

### <In Vivo Examination>

The above indicates that the nerve cells are successfully cultured in vitro in the LASCol-coated group. If LASCol exerted this effect in vivo, LASCol could become a useful pharmaceutical agent for nerve cell regeneration. Thus, the ability of LASCol to enable nerve cell culture in vivo was examined.

### (1) Development of Spinal Cord Injury Model

9-week-old male Sprague-Dawley (SD) rats were used. Each group described below consisted of seven rats. Crush injury was induced by using a standard New York University weight-drop device. The settings of the device were 10 g and 7.5 cm for a drop height. Impact was applied once.

One week after injury, 10 μL of the LASCol solution or phosphate buffered saline (hereinafter simply referred to as “PBS”) was administered to the injured part of the spinal cord. At this time, the temperature of the LASCol solution was at room temperature. Administration was performed by placing the rat on a stereotaxic apparatus and then using a screw-type injector to slowly push a fixed insulin syringe, thereby administering the samples. After allowing the syringe to stand for about two minutes, the needle was withdrawn. This method is the same as a method for performing a standard cell implantation.

For the above-mentioned administration, the practical elastic modulus of the LASCol solution to be injected into the injured part of the spinal cord was measured in advance (rheometer, HAAKE MARS III, Thermo Fisher Scientific Inc.) and was adjusted to 500 Pa to 600 Pa (37° C., pH 7.4). As already described, this value is a value measured on the rheometer 30 minutes after the temperature became 37° C. The practical elastic modulus of 500 Pa is roughly equivalent to the stickiness of honey.

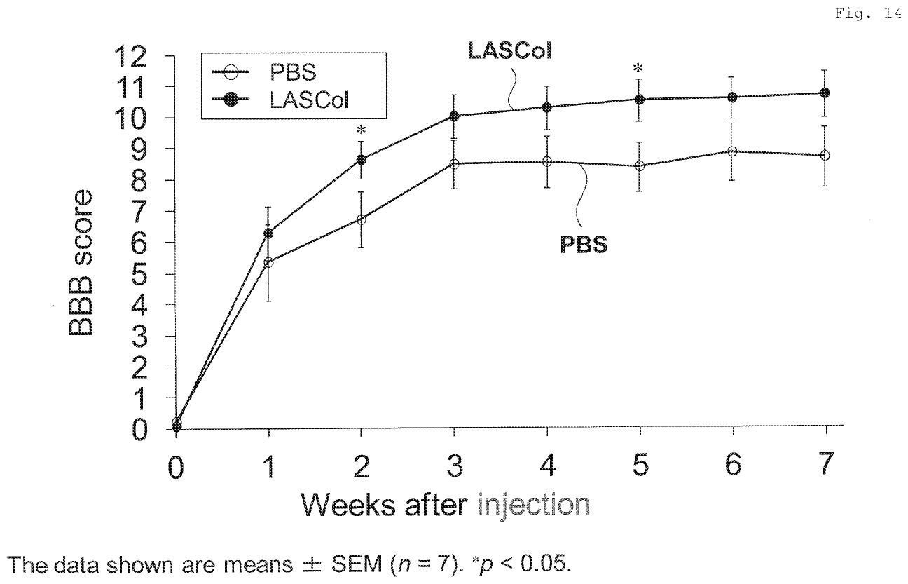

FIG. 14 shows the result of assessment of the state of rats after administration based on a BBB (Basso, Beattie, Bresnahan) locomotor rating scale. Assessment based on the BBB locomotor rating scale was performed by using a 21-point scale assessment (0: complete paralysis to 21: normal). The BBB score was decided by focusing on particularly the state of the hindlimbs. Referring to FIG. 13, the horizontal axis represents time after administration (weeks) and the vertical axis represents the BBB score. A PBS-receiving group (a group that received the above-mentioned PBS) is shown as a white circle and a LASCol-receiving group (a group that received the above-mentioned LASCol solution) is shown as a black circle. The BBB score of 0 represents the heaviest symptom and a higher score represents a healthier state.

The rat recovered rapidly for the first three weeks, and thereafter showed a tendency to recover slowly. The BBB score after five weeks was 11 for the LASCol-receiving group and 9 for the PBS-receiving group. In other words, the LASCol-receiving group recovered better than the PBS-receiving group, with difference in the BBB score being about two. In this context, the score of 9 represents a level at which the rat shows plantar paw placement with weight support when the rat is stationary but not during stepping. The score of 11 represents a level at which the rat shows highly frequent or consistent stepping with weight support. Particularly, significant recovery of the LASCol-receiving group compared with the PBS-receiving group was observed two weeks and five weeks after administration.

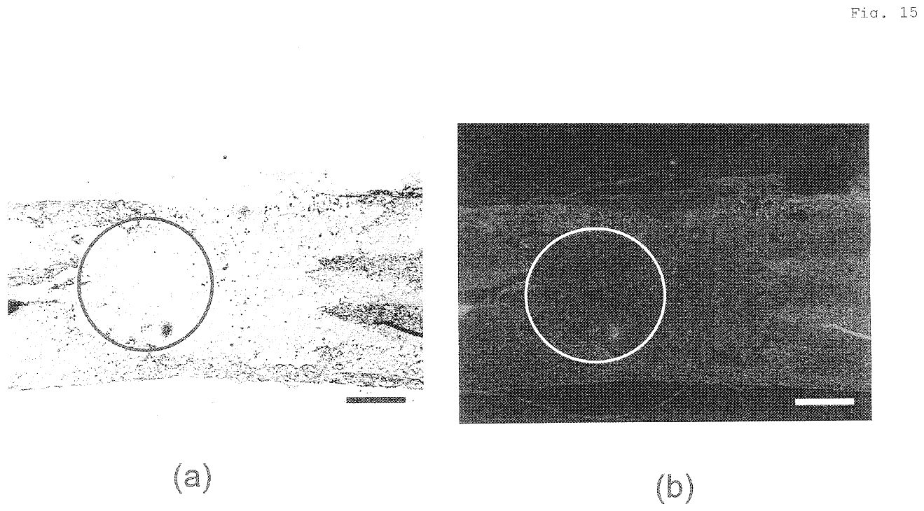

Separately from the experiment for BBB measurement, LASCol was administrated to a six-week-old SD rat immediately after crush injury was induced in the rat, and the tissue of the injured part of the spinal cord was observed eight days after administration. FIG. 15 shows the result of staining of the astrocytes wherein anti GFAP (Glial Fibrillary Acidic Protein) antibody (rabbit polyclonal antibody) was used as a primary antibody.

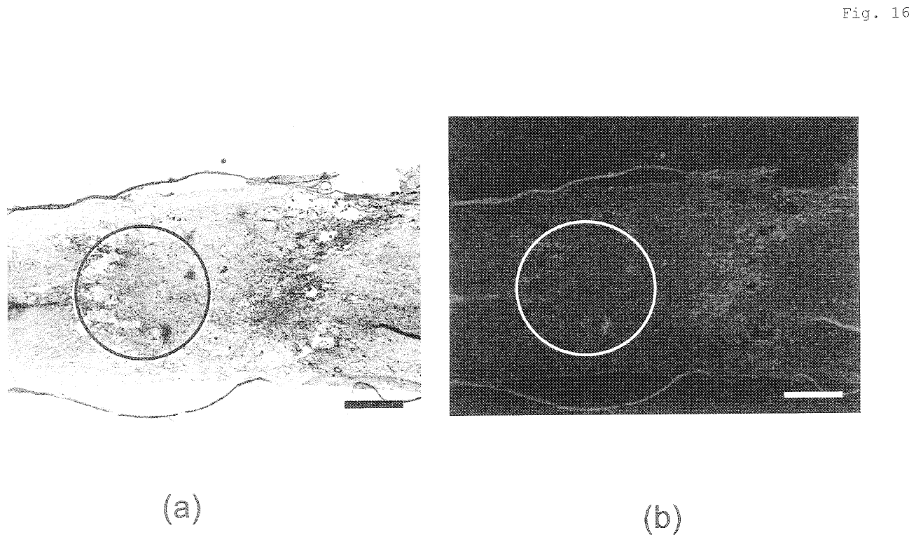

FIG. 16 shows the result of staining of a regenerated nerve wherein anti-phosphorylated GAP-43 (pGAP-43: phosphorylated growth-associated protein 43) antibody (mouse monoclonal antibody) was used as a primary antibody. Phosphorylated GAP-43 is a protein observed when the axon extends. FIG. 15 and FIG. 16 show an identical slice double-stained for GFAP and pGAP-43 and it can be stated that these figures show the same part.

The above-mentioned staining was performed by staining a non-labeled primary antibody with a fluorescently labeled secondary antibody. More specifically, a goat anti-rabbit IgG antibody conjugated to a fluorescent dye (green) excited at a wavelength of 488 nm (CF 488A goat anti-rabbit IgG) was used as the secondary antibody for the anti-GFAP antibody that had been used for staining astrocytes.

A goat anti-mouse IgG antibody conjugated to a fluorescent dye (red) excited at a wavelength of 546 nm (Alexa Fluor 546 goat anti-mouse IgG) was used as the secondary antibody for the anti-phosphorylated GAP-43 antibody. Axio Imager M1 microscope was used as a fluorescent microscope and AxioVision software was used for image acquisition (both were manufactured by Carl Zeiss AG, Tokyo, Japan).

Reference is made to FIG. 15. The scale bar represents 500 μm. FIG. 15(b) shows the result of immunohistochemical staining, wherein 10 μl of LASCol solution (7 mg/ml) was administrated to the injured part immediately after crush injury was induced, the spinal cord was fixed with 4% PFA (paraformaldehyde) eight days after administration, and a 10 μm-thick sagittal slice was taken and subjected to the staining. The staining was performed by using an antibody against GFAP (glial fibrillary acidic protein), which is an astrocyte marker.

In FIG. 15(b), a part stained with the anti-GFAP antibody is stained bright green. A part that is not stained with the anti-GFAP antibody (negative part) represents the injured part. The center of the injured part was encircled by a white circle.

On the other hand, FIG. 15(a) is a photograph obtained by image processing where the part stained with anti-GFAP antibody was blacked and the rest was whitened. Therefore, in FIG. 15(a), the injured part corresponds to a white part within the area encircled by a black circle.

Reference is made to FIG. 16. The scale bar represents 500 μm. FIG. 16(b) shows the image of a serial section in FIG. 15(b) stained with an antibody against phosphorylated GAP43 (pGAP43), which is a marker of a regenerated nerve (an image stained only with pGAP43 of double staining with GFAP and pGAP43). In FIG. 16(b), the part stained with the anti-pGAP43 antibody is stained red. The white circle in FIG. 16(b) corresponds to the same part shown by the white circle in FIG. 15(b).

On the other hand, FIG. 16(a) is a photograph obtained by image processing where the part stained with anti-pGAP43 antibody was blacked and the rest was whitened. The part shown by the white circle in FIG. 16(b) was encircled by a black circle in FIG. 16(a). Therefore, the part encircled by the black circle in FIG. 16(a) corresponds to the same part encircled by the black circle in FIG. 15(a).

Referring to FIG. 15(a) and FIG. 16(a), it can be seen that the GFAP-negative part (white part) in FIG. 15(a) is pGAP43-positive (black part) in FIG. 16(a). In other words, it can be seen that a regenerated axon has extended into the injured part of the spinal cord.

The part where the astrocyte disappeared and became negative for GFAP (white part in FIG. 15(a)) is the injured part. pGAP43 is a protein unique to the regenerated axon of a central nerve. In FIG. 16, presence of the regenerated axon (black part in FIG. 16(a)) was identified in the part with no astrocyte in FIG. 15(a) (injured part: the white part in FIG. 15(a)).

Therefore, it can be concluded that administration of the LASCol solution to the rat with spinal cord injury led to regeneration of the nerve cell in the injured part and recovery.

Next, an effect of LASCol that had been dried and made into a spongy object of a certain shape was examined. Sponge samples that were used were those made by drying different concentrations of LASCol and atelocollagen. The concentrations of LASCol before drying was 30 mg/ml and 50 mg/ml, and the concentration of atelocollagen before drying was 20 mg/ml. The sponge samples are referred to as LA30, LA50, and AC20, respectively. The concentration of each sponge sample before drying is shown in Table 3. Each sponge sample was formed into a shape with a diameter of 2 to 3 mm and a length of 5 mm.

TABLE 3

| NAME OF | | CONCENTRATION |
|---|---|---|
| SPONGE SAMPLES | MATERIAL USED | BEFORE DRYING |
| LA30 | LASCol | 30 mg/ml |
| LA50 | LASCol | 50 mg/ml |
| AC20 | ATELOCOLLAGEN | 20 mg/ml |

A part of the spinal cord (about 5 mm in the vertical direction and about 1 mm in the horizontal direction from the center of the spinal cord) at the level of the eighth to ninth thoracic vertebrae (the same part as the part for crush injury) was excised from a 9-week-old female SD rat. The sponge sample was immediately implanted into the resulting space with tweezers. Each sponge sample was implanted into three rats (the experiment was performed with n=3).

Two weeks after implantation, all the rats were exsanguinated by perfusion with PBS (phosphate buffered saline), and then fixed by perfusion with 4% PFA (perfluoroalkoxy alkane). The spinal cord including the injured part (the part where the sponge sample was implanted) was removed and fixed by immersion in 4% PFA for one day, and after replacing PFA with 30% sucrose (saccharose), was embedded in Surgipath (registered trademark) FSC22 embedding compound (manufactured by Leica Biosystems Inc.). The specimen was horizontally sectioned at a thickness of 10 μm using a cryostat.

After washing the section with PBS, the section was subjected to permeabilization treatment and blocking treatment at room temperature for five minutes using 3% Triton X-100-containing Blocking One Histo (Nacalai Tesque Inc.). A rabbit anti-βIII-tubulin polyclonal antibody (marker of a nerve cell, Abcam plc., ab18207) and a mouse anti-type I collagen monoclonal antibody (for detecting implanted LASCol and AteloCol, Sigma-Aldrich Co., C2456) were used at a 1:200 dilution as primary antibodies, and were reacted with the section at room temperature overnight.

CF488A goat anti-Rabbit IgG (Biotium, Inc.) and Alexa Fluor 555 goat anti-mouse IgG (Thermo Fisher Scientific Inc.) were used at a 1:200 dilution as secondary antibodies and were reacted with the section at room temperature for 30 minutes. The nuclei of the cells were stained with 0.3 μM DAPI. After putting a cover glass on the section by using Fluoromount/Plus (Diagnostic BioSystems Inc.), a fluorescent microscope, specifically Axio Imager M1 microscope with AxioVision software (Carl Zeiss AG) was used to observe the section and acquire image data thereof.

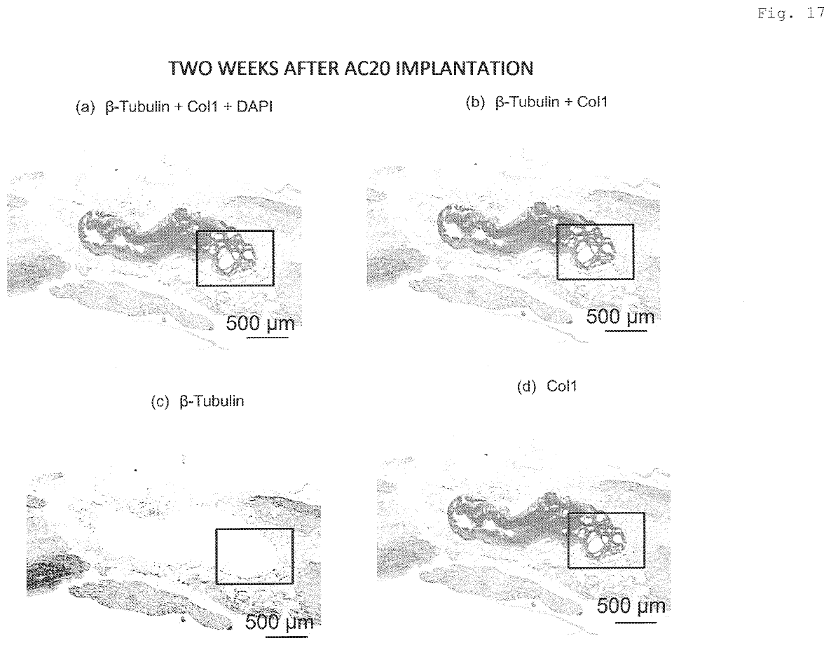

FIG. 17 shows the photographs taken two weeks after the AC20 implantation. FIG. 17(a) is a merged photograph of staining with β-Tubulin (staining of an axon), Col1 (staining of collagen), and DAPI (staining of a cell nucleus). FIG. 17(b) is a merged photograph of staining with β-Tubulin (staining of an axon) and Col1 (staining of collagen). FIG. 17(c) is a photograph only of staining with β-Tubulin (staining of an axon) and FIG. 17(d) is a photograph only of staining with Col1 (staining of collagen).

The part in the center of FIG. 17(d) that looks black corresponds to the sponge sample AC20. Even two weeks after implantation, the sponge sample AC20 kept a well-defined shape as is seen in the figure. Furthermore, in FIG. 17(c), there was no stained area in the part shown in black in FIG. 17(d). This means that a neural axon did not extend into the sponge sample AC20 made of atelocollagen.

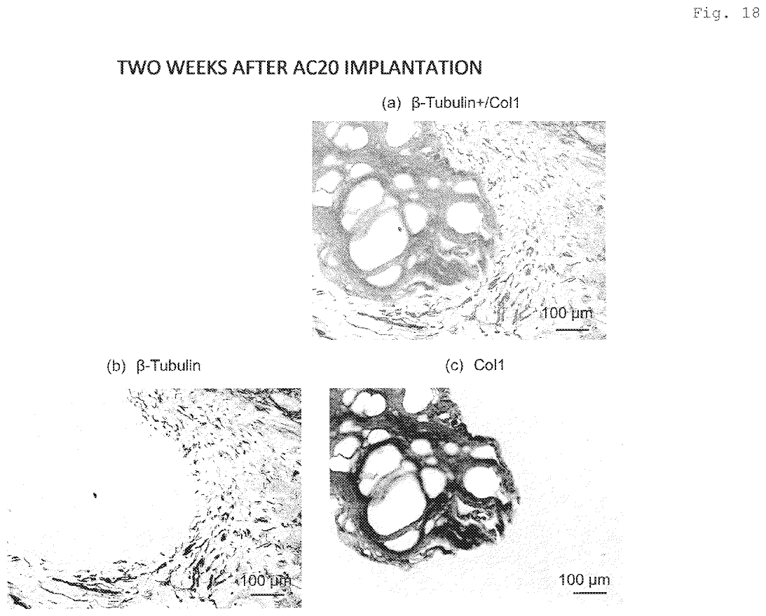

FIG. 18 includes enlarged views of a boxed area in respective photographs in FIG. 17. FIG. 18(a) is a merged photograph of staining with β-Tubulin (staining of an axon) and Col1 (staining of collagen). FIG. 18(b) is a photograph only of staining with β-Tubulin (staining of an axon) and FIG. 18(c) is a photograph only of staining with Col1 (staining of collagen). The sponge sample made of atelocollagen shown as a black reticulated structure in FIG. 18(c) and the neural axon shown in black in FIG. 18(b) had no overlapping area. In other words, even when the photograph was enlarged and observed, no evidence was found for extension of the neural axon into the sponge sample AC20 made of atelocollagen.

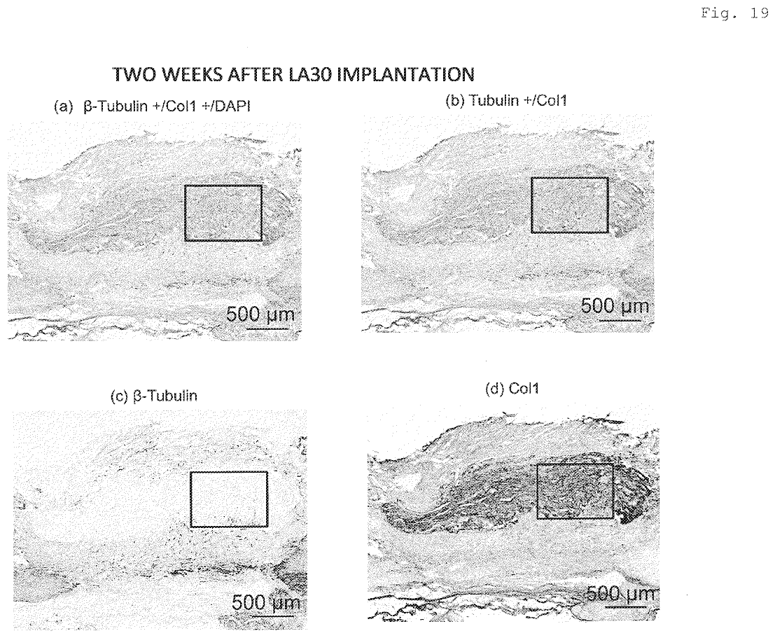

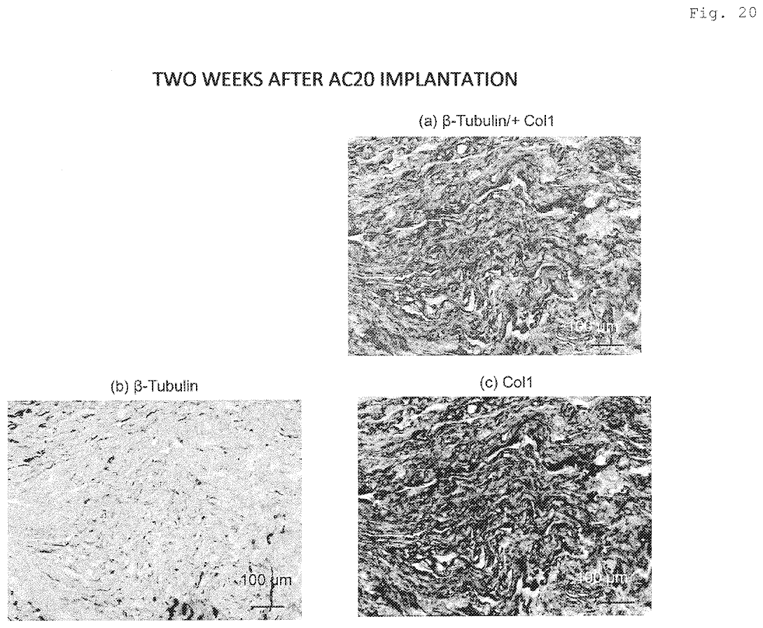

FIG. 19 and FIG. 20 are photographs taken when the sponge sample LA30 was implanted. FIG. 19(a) is a merged photograph of staining with β-Tubulin (staining of an axon), Col1 (staining of collagen), and DAPI (staining of a cell nucleus). FIG. 19(b) is a merged photograph of staining with β-Tubulin (staining of an axon) and Col1 (staining of collagen). FIG. 19(c) is a photograph only of staining with β-Tubulin (staining of an axon) and FIG. 19(d) is a photograph only of staining with Col1 (staining of collagen).

FIG. 20 includes enlarged photographs of boxed areas in FIG. 19. FIG. 20(a) is a merged photograph of staining with β-Tubulin (staining of an axon) and Col1 (staining of collagen). FIG. 20(b) is a photograph only of staining with β-Tubulin (staining of an axon) and FIG. 20(c) is a photograph only of staining with Col1 (staining of collagen).

The dark part in the center of FIG. 19(d) corresponds to the LA30. Even two weeks after implantation, the sponge sample LA30 kept the shape thereof to such an extent that the sponge sample could be identified in the photograph of staining. In FIG. 19(c) and FIG. 20(b), which are images of neural axon staining, staining of the neural axon was observed at a location where LA30 was present.

In particular, in FIG. 20(b), a black stained part was clearly observed in the sponge sample.

These findings demonstrated that the neural axon had extended into the implanted LASCol sponge sample.

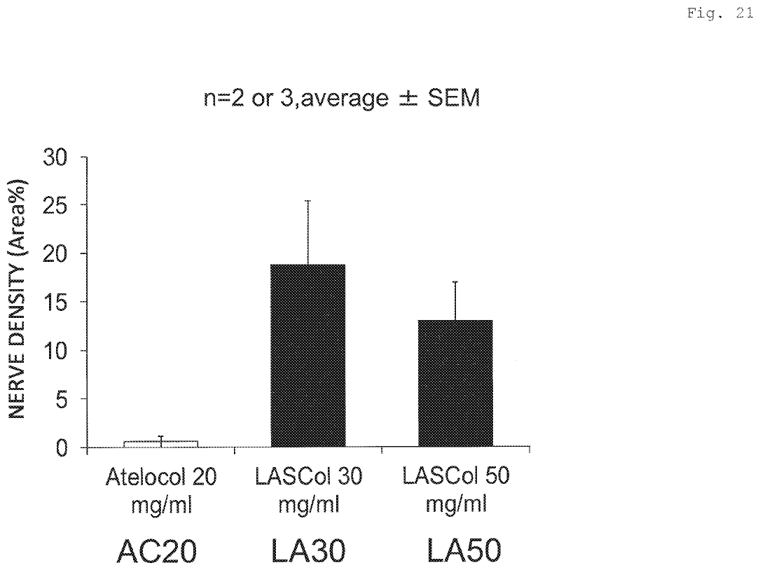

FIG. 21 shows the amount of the neural axons in the implanted sponge sample, which is expressed as a percentage of the area occupied by β-tubulin relative to the area where Col1 is present (nerve density (Area %)).

The horizontal axis represents the type of the sponge samples. In the case of AC20, the atelocollagen sponge sample, little β-Tubulin was present in Col1. In contrast, in the case of the LASCol sponge sample, the axon was found in the sponge sample. Furthermore, when the LASCol concentrations of 30 mg/ml and 50 mg/ml were compared, the axon area was larger for the concentration of 30 mg/ml (LA30).

A higher LASCol concentration before drying results in a denser sponge sample. Therefore, the result of FIG. 21 indicates that a sponge LASCol having a structure with moderately small vacant spaces is favorable for extension of the axon of the regenerated nerve.

It was found from the above that LASCol (gel and a dry product) could be favorably used as the therapeutic agent for nerve damage. Because a nerve cell has a similar property regardless of the place where the nerve cell exists, LASCol can be used favorably as a therapeutic agent for injury of spinal cord, which is a central nerve, but also the therapeutic agent for damage of nerve including a peripheral nerve.

### INDUSTRIAL APPLICABILITY

The nerve cell culture material according to the present invention can be favorably used as a scaffold material or an additive for culturing nerve cells. The therapeutic agent for nerve damage according to the present invention can also be used favorably for regenerative treatment of a damaged part of a nerve severed due to spinal cord injury. Furthermore, the inventive therapeutic agent can be used as a therapeutic agent for regeneration of a nerve cell in other places.

1. A nerve cell culture material comprising LASCol.

2. The nerve cell culture material according to claim 1, for suppressing growth of glial cells.

3. The nerve cell culture material according to claim 1, wherein the LASCol contains a degradation product of collagen or atelocollagen in which a chemical bond between Y₁ and Y₂ of an α1 chain is cleaved in an amino-terminal amino acid sequence including a triple helical domain of the collagen or atelocollagen, the sequence being shown by the following (A), or a chemical bond between G and X₃ of an α2 chain is cleaved in an amino-terminal amino acid sequence including a triple helical domain of the collagen or atelocollagen, the sequence being shown by the following (B),
(SEQ ID NO: 1)
(A)-Y₁-Y₂-Y₃-G-Y₄-Y₅-G-Y₆-Y₇-G-Y₈-Y₉-G-
(where G represents glycine, and Y₁ to Y₉ each represent an optional amino acid),

| | |
|---|---|
| (B)-G-X₁-X₂-G-X₃-X₄-G-X₅-X₆-G- | (SEQ ID NO: 2) |

(where G represents glycine, and X₁ to X₆ each represent an optional amino acid).

4. A therapeutic agent for nerve damage comprising LASCol.

5. The therapeutic agent for nerve damage according to claim 4, wherein the LASCol is dried.

6. The therapeutic agent for nerve damage according to claim 4, for suppressing growth of glial cells.

7. The therapeutic agent for nerve damage according to claim 4, wherein the LASCol contains a degradation product of collagen or atelocollagen in which a chemical bond between Y₁ and Y₂ of an α1 chain is cleaved in an amino-terminal amino acid sequence including a triple helical domain of the collagen or atelocollagen, the sequence being shown by the following (A), or a chemical bond between G and X₃ of an α2 chain is cleaved in an amino-terminal amino acid sequence including a triple helical domain of the collagen or atelocollagen, the sequence being shown by the following (B),
(SEQ ID NO: 1)
(A)-Y₁-Y₂-Y₃-G-Y₄-Y₅-G-Y₆-Y₇-G-Y₈-Y₉-G-
(where G represents glycine, and Y₁ to Y₉ each represent an optional amino acid),

| | |
|---|---|
| (B)-G-X₁-X₂-G-X₃-X₄-G-X₅-X₆-G- | (SEQ ID NO: 2) |

(where G represents glycine, and X₁ to X₆ each represent an optional amino acid).

8. The therapeutic agent for nerve damage according to claim 4, wherein the nerve is the spinal cord.
